(19) United States
(12) Patent Application Publication
(10) Pub. No.: US 2019/0091248 A1
(43) Pub. Date: Mar. 28, 2019

(54) AGENT FOR INCREASING INTESTINAL BUTYRIC ACID AND PROLIFERATION AGENT FOR BUTYRIC ACID-PRODUCING BACTERIA

(71) Applicant: B FOOD SCIENCE CO., LTD., Chita-shi, Aichi (JP)

(72) Inventors: Takumi TOCHIO, Chita-shi, Aichi (JP); Kenta KONISHI, Chita-shi, Aichi (JP); Saki NAKAMURA, Chita-shi, Aichi (JP)

(21) Appl. No.: 16/084,310

(22) PCT Filed: Mar. 14, 2017
(86) PCT No.: PCT/JP2017/010066

§ 371 Date: Sep. 12, 2018

(30) Foreign Application Priority Data
Mar. 14, 2016 (JP) 2016-049561

(51) Int. Cl. A61K 31/702; A61P 1/04; A61P 3/04; A61P 3/10; A61P 35/00; A23L 33/21

(52) U.S. Cl. CPC A61K 31/702; A61P 1/04; A61P 3/04; A61P 3/10; A61P 35/00; A23V 2200/332; A23V 2002/00; A23V 2250/284; A23V 2200/308; A23V 2200/32; A23V 2200/328; A23L 33/21

(57) ABSTRACT

[Problem]

To provide: an agent for increasing intestinal butyric acid which is capable of effectively increasing intestinal butyric acid; a food composition for increasing intestinal butyric acid; a proliferation agent for butyric acid-producing bacteria; a food composition for proliferating butyric acid-producing bacteria; a method for increasing intestinal butyric acid; a method for proliferating butyric acid-producing bacteria; a method for treating or preventing intestinal inflammation, fatty liver, diabetes, colorectal cancer or obesity using the same; and use of 1-kestose for producing a pharmaceutical preparation for treatment or prevention of these diseases.

[Solution]

An agent for increasing intestinal butyric acid and a proliferation agent for butyric acid-producing bacteria, each comprising 1-kestose as an active ingredient. According to the present invention, intestinal butyric acid in humans or animals can be easily and effectively increased with little side effects or safety concerns. According to the present invention, furthermore, intestinal inflammation, fatty liver, diabetes, colorectal cancer or obesity can be prevented or treated by increasing intestinal butyric acid in humans or animals.

### TECHNICAL FIELD

The present invention relates to an agent for increasing intestinal butyric acid, a food composition for increasing intestinal butyric acid, a proliferation agent for butyric acid-producing bacteria, a food composition for proliferating butyric acid-producing bacteria, a method for increasing intestinal butyric acid, a method for proliferating butyric acid-producing bacteria, and a method for treatment or prevention of intestinal inflammation, fatty liver, diabetes, colorectal cancer, or obesity and use of 1-kestose for producing a pharmaceutical preparation for treatment or prevention of these diseases.

### BACKGROUND ART

Butyric acid is a short chain fatty acid and produced by butyric acid-producing bacteria that reside in the intestines in human and animal bodies. Studies in recent years have reported that butyric acid has various physiological effects such as anti-inflammatory effects and colorectal cancer preventing effects and that butyric acid may prevent or ameliorate various diseases.

Based on theses, attempts to use butyric acid have been made for prevention and treatment of diseases and health promotion. Meanwhile, butyric acid is a substance having a strong bad smell and therefore butyric acid itself is difficult to be added to and used in pharmaceutical preparations and foods. Therefore, attempts to increase the amount of butyric acid in human and animal intestines have been made. For example, Patent Literature 1 discloses a food composition having the effect of increasing the intestinal butyric acid concentration and comprising D-mannitol or D-sorbitol as an active ingredient and Patent Literature 2 discloses an agent for increasing intestinal butyric acid-producing bacteria, comprising Bacillus subtilis cells as an active ingredient.

### CITATION LIST

### Patent Literature

### Patent Literature 1:

Japanese Patent Laid-Open No. 2004-49093

### Patent Literature 2:

Japanese Patent Laid-Open No. 2013-147469

### SUMMARY OF INVENTION

### Technical Problem

However, no substance or method capable of effectively increasing intestinal butyric acid has been sufficiently provided and development of such a substance or method has been desired. The present invention was made to solve the problem and an object of the present invention is to provide an agent for increasing intestinal butyric acid, a food composition for increasing intestinal butyric acid, a proliferation agent for butyric acid-producing bacteria, a food composition for proliferating butyric acid-producing bacteria, a method for increasing intestinal butyric acid, and a method for proliferating butyric acid-producing bacteria that are capable of effectively increasing intestinal butyric acid and a method for treatment or prevention of intestinal inflammation, fatty liver, diabetes, colorectal cancer, or obesity using one of these and use of 1-kestose for producing a pharmaceutical preparation for treatment or prevention of these diseases.

### Solution to Problem

The present inventors have found, as a result of diligent studies, that 1-kestose increases the amount and the concentration of intestinal butyric acid and that 1-kestose increases the number of intestinal butyric acid-producing bacteria. Accordingly, based on these findings, the following inventions were completed.

(1) An agent for increasing intestinal butyric acid according to the present invention comprises 1-kestose as an active ingredient.
(2) An agent for increasing intestinal butyric acid according to the present invention is preferably used to let a human or an animal take 0.04 g/kg body weight or more of 1-kestose per day.
(3) An agent for increasing intestinal butyric acid according to the present invention can be used for prevention or treatment of intestinal inflammation, fatty liver, diabetes, colorectal cancer, or obesity.
(4) A food composition for increasing intestinal butyric acid according to the present invention comprises 1-kestose as an active ingredient.
(5) A proliferation agent for butyric acid-producing bacteria according to the present invention comprises 1-kestose as an active ingredient.
(6) A food composition for proliferating butyric acid-producing bacteria according to the present invention comprises 1-kestose as an active ingredient.
(7) A method for increasing intestinal butyric acid according to the present invention comprises the step of increasing the amount or the concentration of butyric acid in intestines in a human or an animal by letting the human or the animal take 1-kestose.
(8) The first aspect of the method for proliferating butyric acid-producing bacteria according to the present invention comprises the step of increasing the number of butyric acid-producing bacteria in intestines in a human or an animal by letting the human or the animal take 1-kestose.
(9) The second aspect of the method for proliferating butyric acid-producing bacteria according to the present invention comprises the step of adding 1-kestose to a medium containing butyric acid-producing bacteria to culture the butyric acid-producing bacteria.
(10) A method for treatment or prevention of intestinal inflammation, fatty liver, diabetes, colorectal cancer, or obesity according to the present invention comprises the step of increasing the amount or the concentration of butyric acid or the number of butyric acid-producing bacteria in intestines in a human or an animal having or being at risk of having intestinal inflammation, fatty liver, diabetes, colorectal cancer, or obesity by letting the human or the animal take 1-kestose.
(11) Use of 1-kestose according to the present invention is use of 1-kestose for producing a pharmaceutical preparation for treatment or prevention of intestinal inflammation, fatty liver, diabetes, colorectal cancer, or obesity.

### Advantageous Effects of Invention

1-kestose is an oligosaccharide and a substance that is contained in vegetables such as onion and garlic, and in cereals such as barley and rye and has been present in foods since ancient times. Moreover, no toxicity of 1-kestose has been found in any of mutagenicity, acute toxicity, subchronic toxicity, and chronic toxicity tests. From these, 1-kestose is considered to be very safe (Food processing and ingredients, Vol. 49, No. 12, p. 9, 2014). Moreover, since 1-kestose is highly water-soluble and have a good sweet taste quality similar to sugar, it can be taken in easily on a daily basis as it is or as a sweetener or the like and can be easily formulated into various foods, pharmaceutical preparations and the like.

Accordingly, intestinal butyric acid in humans and animals can be easily and effectively increased with little concerns about side effects and safety, according to the present invention. Moreover, according to the present invention, intestinal inflammation, fatty liver, diabetes, colorectal cancer, or obesity can be prevented or treated by increasing intestinal butyric acid in a human or an animal.

### BRIEF DESCRIPTION OF DRAWINGS

FIG. 1 is a table illustrating the concentrations and the amounts of short chain fatty acids (acetic acid, isovaleric acid, and valeric acid) in cecal contents in rats (Groups A to E) given 0 to 1.34 g/kg body weight of 1-kestose.

FIG. 2 is a set of bar graphs illustrating the concentrations and the amounts of short chain fatty acids (butyric acid, propionic acid, and isobutyric acid) in cecal contents in rats (Groups A to E) given 0 to 1.34 g/kg body weight of 1-kestose.

FIG. 3 is a set of bar graphs illustrating the concentrations and the amounts of short chain fatty acids (acetic acid, isovaleric acid, and valeric acid) in cecal contents in rats (Groups A to E) given 0 to 1.34 g/kg body weight of 1-kestose.

FIG. 4 (I) is a table illustrating the means, standard deviations, and significant difference of the copy number of 16S rDNA from various microorganisms in cecal contents in rats given no 1-kestose (Group A) and rats given 1-kestose (Group E). FIG. 4 (II) is a bar graph illustrating the means and standard deviations of the copy number of the 16S rDNA.

### DESCRIPTION OF EMBODIMENTS

Hereinafter, an agent for increasing intestinal butyric acid, a food composition for increasing intestinal butyric acid, a proliferation agent for butyric acid-producing bacteria, a food composition for proliferating butyric acid-producing bacteria, a method for increasing intestinal butyric acid, a method for proliferating butyric acid-producing bacteria, and a method for treatment or prevention of intestinal inflammation, fatty liver, diabetes, colorectal cancer, or obesity and use of 1-kestose for producing a pharmaceutical preparation for treatment or prevention of these diseases according to the present invention will be described in detail.

“Butyric acid” is a linear carboxylic acid that is also referred to as butanoic acid or n-butyric acid, and has a molecular formula of C₄H₈O₂ and a rational formula of CH₃(CH₂)₂COOH. Butyric acid is known to be produced by butyric acid-producing bacteria habitually residing in the intestines in humans and animals.

Here, the “butyric acid-producing bacteria” in the present invention are microorganisms that produce butyric acid. Specific examples of such microorganisms include bacteria included in Clostridium cluster-XIVa such as Eubacterium Hallii, Butyrivibrio crossotus, Coprococcus eutactus, Coprococcus catus, Clostridium symbiosum, and Roseburia cecicola; bacteria included in Clostridium cluster-XVI such as Clostridium innocuum, Eubacterium tortuosum, and Eubacterium cylindroides; bacteria included in Clostridium cluster-I such as Clostridium butyricum, Eubacterium moniliforme, and Clostridium acetobutylicum; bacteria included in Clostridium cluster-IV such as Clostridium sporosphaeroides and Eubacterium sp. A2-207; and bacteria included in Clostridium cluster-XV such as Eubacterium barkeri and Eubacterium limosum.

“1-kestose” is an oligosaccharide that is a trisaccharide composed of 1 molecule of glucose and 2 molecules of fructose. 1-kestose can be obtained by an enzymatic reaction of sucrose as a substrate with an enzyme such as one disclosed in Japanese Patent Laid Open No. 58-201980. Specifically, β-fructofuranosidase is added to a sucrose solution and the enzymatic reaction is performed by leaving the mixture at 37° C. to 50° C. for around 20 hours to obtain a reaction solution containing 1-kestose. This reaction solution containing 1-kestose is purified by separating 1-kestose and other sugars (glucose, fructose, sucrose, oligosaccharides composed of 4 or more sugars) using the chromatographic separation disclosed in Japanese Patent Laid Open No. 2000-232878 to obtain a high purity 1-kestose solution. 1-kestose can be obtained as crystals by subsequently concentrating this high purity 1-kestose solution and then crystallizing 1-kestose by a crystallization process such as one disclosed in Japanese Patent Publication No. 6-70075.

Moreover, 1-kestose is contained in commercially available fructo-oligosaccharides and therefore these may be used as they are or after separating and purifying 1-kestose from the fructo-oligosaccharides by the aforementioned method. Accordingly, 1-kestose-containing compositions such as oligosaccharides containing 1-kestose may be used as the 1-kestose of the present invention. When a 1-kestose-containing composition is used, the purity of the 1-kestose is preferably 80% by mass or more, more preferably 85% by mass or more, and further preferably 90% by mass or more. The “purity” of 1-kestose in the present invention is percent by mass of 1-kestose when taking the total amount of the sugars as 100%.

1-kestose can be used by letting a human or an animal take the 1-kestose. An example of the intake (dose) of 1-kestose is 0.04 g/kg body weight or more per day. Such an intake may be taken once a day or divided into multiple doses. As illustrated in Example 1 described below, such an intake can markedly increase the concentration and the amount of intestinal butyric acid.

1-kestose functions to increase the amount and the concentration of butyric acid in the intestines in humans and animals. Moreover, 1-kestose functions to increase the number of butyric acid-producing bacteria in the intestines in humans and animals. Accordingly, the method for letting a human or animal take 1-kestose by may be any method, as long as it is a method that allows 1-kestose to reach the intestine thereof. Specific examples of the method include letting a human or an animal take 1-kestose as it is or in the form of beverage or food or a pharmaceutical preparation. Other examples include administering 1-kestose from anus directly or through an inserted tube, adding 1-kestose to an enteral nutrition agent and administering that by enteral nutrition via a tube inserted into a gastrointestinal tract such as the stomach or the small intestine, and the like.

In addition, 1-kestose can be used in vitro to proliferate butyric acid-producing bacteria. In this case, for example, a method adding 1-kestose to a medium containing butyric acid-producing bacteria to culture the butyric acid-producing bacteria in predetermined culture conditions suitable for the butyric acid-producing bacterium is available. Specific examples include a method inoculating Clostridium butyricum, when Clostridium butyricum is used as a butyric acid-producing bacterium, into a medium containing 2% by weight of 1-kestose as a carbon source, 2% by weight of an amino acid solution as a nitrogen source, 0.75% by weight of calcium carbonate and anaerobically culturing the butyric acid-producing bacterium at 35 to 37° C. for 16 to 24 hours.

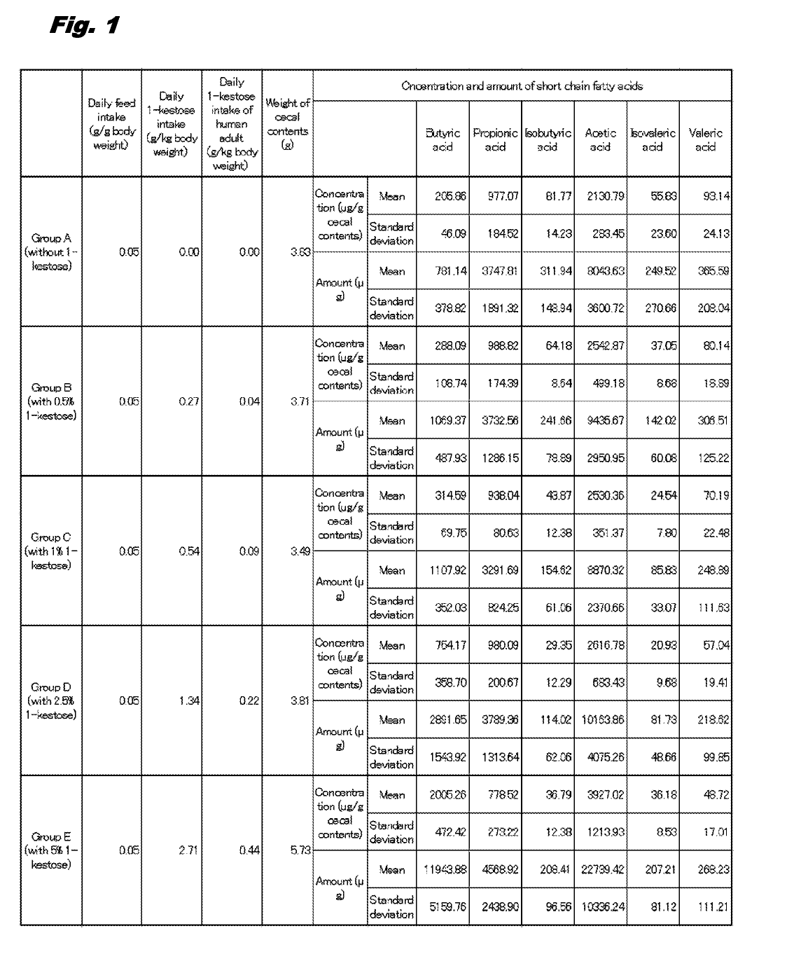

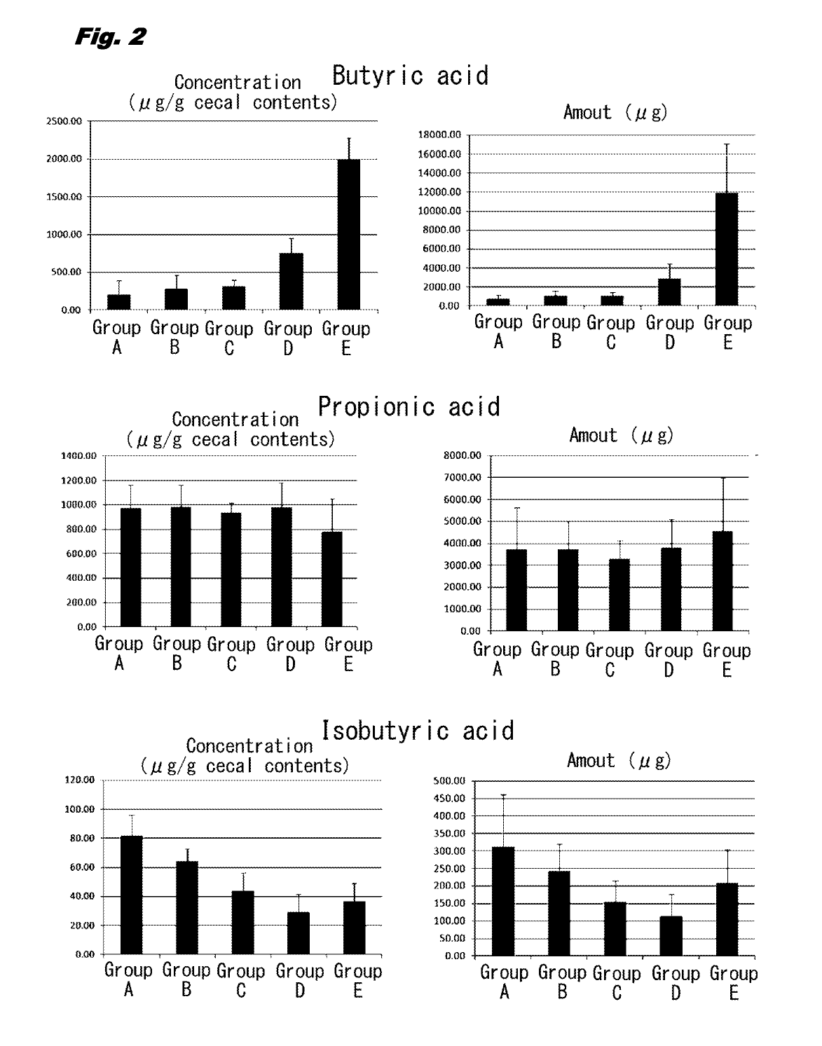

Butyric acid has been confirmed to exhibit, in ulcerative colitis model rats induced by dextran sulfate sodium (DSS), a significant inflammatory repair effect by injection into the intestine. Moreover, it has been confirmed that the oral administration of a spore formulation of Clostridium butyricum M588, a butyric acid-producing bacterium, has a prominent effect of reducing inflammation in the same model rat. Furthermore, butyric acid injection therapy is provided for human ulcerative colitis (Journal of intestinal microbiology, vol. 19(1), p. 1-8, 2005; right column on page 2, FIGS. 4-1 and 4-2, etc.).

Therefore, intestinal inflammation can be prevented or treated by increasing the concentration or the amount of butyric acid or the number of butyric acid-producing bacteria in the intestines by letting a human or an animal take 1-kestose. Moreover, 1-kestose can be used to produce a pharmaceutical preparation for treatment or prevention of intestinal inflammation.

Here, “intestinal inflammation” in the present invention means conditions with inflammation in an intestinal tract and includes disease conditions. Examples of diseases developed from intestinal inflammation include inflammatory bowel disease. The term inflammatory bowel disease may narrowly refer ulcerative colitis and Crohn disease or generally refer any enteric inflammatory disease (Integrated handbook of internal medicine (Progress 8), Gastrointestinal Diseases, pp. 320, 1997, Nakayama Shoten Co., Ltd.). The inflammatory bowel disease in the present invention means the later, any inflammatory disease in an intestinal tract. The present invention may be preferably used in prevention or treatment of intestinal inflammation excluding allergy.

Moreover, oral administration of butyric acid to rats at 0.02 g/kg body weight per day has been shown to suppress fatty degeneration and inflammation in the liver by the intake of high-fat meals and to normalize the values of AST (aspartate aminotransferase), ALT (alanine aminotransferase), cholesterol, LDL (low-density lipoprotein) in blood, neutral fat and fasting blood sugar levels, and an insulin resistance index (HOMA-IR) (G. Mattace Raso et al., PLoS ONE, vol. 8, no. 7, e68626, 2013, Abstract, Result, etc.). Therefore, fatty liver can be prevented or treated by increasing the concentration or the amount of butyric acid or the number of butyric acid-producing bacteria in the intestines by letting a human or an animal take 1-kestose. Moreover, 1-kestose can be used to produce a pharmaceutical preparation for treatment or prevention of fatty liver.

Moreover, intraperitoneal administration of butyric acid to a childhood diabetes model rat at 500 mg/kg of body weight per day (in terms of the dose of sodium butyrate) has been shown to decrease β cell death, improve the function and proliferation of β cells, and improve the glucose homeostasis (S. Khan et al., Chem. Biol. Interact., vol. 213, p. 1-12, 2014, Abstract, etc.). Therefore, diabetes can be prevented or treated by increasing the concentration or the amount of butyric acid or the number of butyric acid-producing bacteria in the intestines by letting a human or an animal take 1-kestose. Moreover, 1-kestose can be used to produce a pharmaceutical preparation for treatment or prevention of diabetes.

Moreover, by addition of butyric acid to the culture of LIM1215 at final concentration of 1 mmol/L, human colorectal cancer cells has been shown to extend the proliferation time and decrease the proliferation rate (Whitehead R H et al., Gut 27, p. 1457-1463, 1986, SUMMARY, etc.). Furthermore, it has been reported that butyric acid suppresses the development of colorectal cancer by suppressing abnormal proliferation of mucosa epithelial cells in the large intestine, promoting the cell death and differentiation of mutated cells, suppressing the transcription of abnormal proteins, or suppressing the development of aberrant crypt foci (ACF) which is a lesion in the early period of colorectal cancer (Journal of intestinal microbiology, vol. 16(1), p. 35-42, 2002, left column on p. 40 and Table 5, etc.). Therefore, colorectal cancer can be prevented or treated by increasing the concentration or the amount of butyric acid or the number of butyric acid-producing bacteria in the intestines by letting a human or an animal take 1-kestose. Moreover, 1-kestose can be used to produce a pharmaceutical preparation for treatment or prevention of colorectal cancer.

Moreover, oral administration of butyric acid at 5 g/kg body weight per day (in terms of the dose of sodium butyrate) has been shown to reduce body weight and the body fat percentage in mice in which obesity has been induced by high-fat meals and to suppress weight gain and increase of the body fat percentage by eating of high-fat meals in mice that are not obese (Zhanguo Gao et. al., DIABETES, VOL. 58, July 2009, p. 1509-1517; FIGS. 1 E and F and FIGS. 7 A and B, etc.). Similarly, it has been also shown that increase of body weight and increase of the body fat percentage are suppressed in mice fed on high-fat meals into which 5% by mass of sodium butyrate is mixed (Hua V. Lin et. al., PLoS ONE, Vol. 7, Issue 4, e35240, April 2012, FIG. 1 A, etc.). Therefore, obesity can be prevented or treated by increasing the concentration or the amount of butyric acid or the number of butyric acid-producing bacteria in the intestines by letting take a human or an animal 1-kestose. Moreover, 1-kestose can be used to produce a pharmaceutical preparation for treatment or prevention of obesity.

Examples of specific aspects of the agent for increasing intestinal butyric acid and the proliferation agent for butyric acid-producing bacteria according to the present invention include pharmaceutical preparations and quasi drugs, food additives, and health foods such as supplements.

The dosage form of the pharmaceutical preparations, quasi drugs, and supplements containing 1-kestose are not particularly limited, and a dosage form suitable for the mode of administration can be selected as appropriate. For example, when orally administered, the dosage form may be a solid or liquid dosage form such as a powder, a tablet, a dragee, a capsule, a granule, a dry syrup, a solution, a syrup, a drop, and a health drink.

The aforementioned dosage forms of pharmaceutical preparations, quasi drugs, and supplements can be prepared by methods known to those skilled in the art. For example, for a powder, 800 g of 1-kestose and 200 g of lactose are mixed well and then the mixture is wetted by adding 300 mL of 90% ethanol. Subsequently, the wet powder is granulated, dried with ventilation at 60° C. for 16 hours, and then sized to obtain 1000 g of a suitable size of powder (1-kestose content 800 mg/l g). For tablets, 300 g of 1-kestose, 380 g of powdery reduced starch syrup, 180 g of rice starch, and 100 g of dextrin are mixed well and then the mixture is wetted by adding 300 mL of 90% ethanol. Subsequently, the wet powder is extruded and granulated and then dried with ventilation at 60° C. for 16 hours to obtain granules. Then, these granules are sized using a 850 μm sieve, 50 g of a sucrose fatty acid ester was subsequently added to and mixed with 470 g of the sized granules, and then the mixture was pressed into tablets with a rotary tableting machine (6B-2, manufactured by Kikusui Seisakusho Ltd.) to obtain 5000 tablets having a diameter of 8 mm and a weight of 200 mg (1-kestose content 60 mg/tablet).

Examples of specific aspects of the food composition for increasing intestinal butyric acid and the food composition for proliferating butyric acid-producing bacteria according to the present invention include beverages, dairy products, granules for eating, pastes, flavoring agents, retort pouches, baby foods, fermented foods, preserved foods, processed foods such as processed marine products, processed meat products, and processed grain products, food additives, health foods, and animal feed.

1-kestose can be used by adding to various foods and drinks, food additives, and animal feed in their normal production process. Since 1-kestose has a degree of sweetness of 30 and its quality of taste, physical properties, and workability are close to those of sucrose, it can be used like sugar in the production process of various foods and drinks, for example, by replacing a part or all of sugar with 1-kestose, and various foods and drinks, food additives, and animal feed can be produced.

The present invention will be described with reference to Examples below. The technical scope of the present invention is not limited by features illustrated by these Examples. In the following Examples, compositions containing a predetermined purity of 1-kestose are referred to as “1-kestose”.

### EXAMPLES

### <Example 1> Study on Amount of Intestinal Short Chain Fatty Acid Upon Intake of 1-Kestose

(1) Breeding of Rats with Giving Oral Intake of 1-Kestose

The feeds containing 0, 0.5, 1.0, 2.5, and 5% by mass of 1-kestose were prepared by CLEA Japan, Inc. on commission. The composition of the feeds is described below. 40 SD rats (Japan SLC, Inc.) were divided into 5 groups of 8 animals and designated as Groups A to E. The rats were maintained for 30 days with ad libitum feeding of the feed without 1-kestose to Group A and of the ones with 1-kestose to Groups B to E, respectively. The rats were maintained under conditions at 23±1° C. in temperature with 12 hours of light periods (from 8:00 to 20:00) and 12 hours of dark periods (from 20:00 to 8:00).

[Composition (in % by mass) of feeds] Cornstarch 39.7486, milk casein 20, pregelatinized cornstarch 13.2, granulated sugar and/or 1-kestose (99% by mass in purity, B Food Science Co., Ltd.) 10, purified soybean oil 7, cellulose powder 5, mineral mixture 3.5, vitamin mixture 1, L-cystine 0.3, choline bitartrate 0.25, tert-butylhydroquinone 0.0014.

### (2) Study on Amount of Intestinal Short Chain Fatty Acid

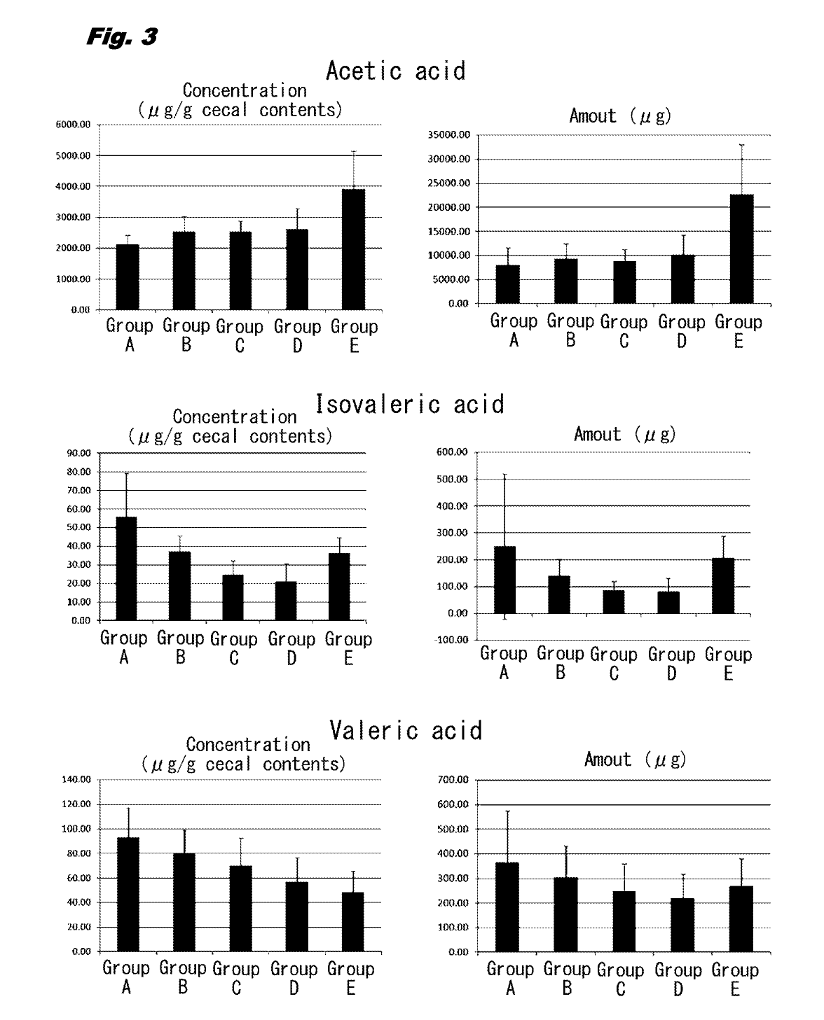

The rats in each group described in Example 1 (1) were dissected and their ceca were extracted. The contents of the ceca were collected and weighed. The concentrations of short chain fatty acids (butyric acid, propionic acid, isobutyric acid, acetic acid, isovaleric acid, and valeric acid) in the cecal contents were measured with a gas chromatography-mass spectrometer (GC/MS). The amounts of the short chain fatty acids in the cecal contents were calculated from the results of the measurement of the short chain fatty acid concentrations. Subsequently, the means of concentrations and amounts of short chain fatty acids for each group were calculated and shown in bar graphs. The results are illustrated in FIGS. 1 to 3.

The intake of a drug in rat has been reported to be converted into a corresponding human adult intake by the formula 1 below (Paragraph [0065] in Japanese Patent Laid Open No. 2014-526521, Shannon Reagan-Shaw et. al., The FASEB Journal, Vol. 22, March 2007, p. 659-661). Therefore, the daily intake of 1-kestose in rat was converted into corresponding human adult intake by the formula 1. The results were also set forth in FIG. 1.

daily intake of 1-kestose in human adult (g/kg body weight)=daily intake of 1-kestose in rat (g/kg body weight)×6/37 [Formula 1]

As illustrated in FIGS. 1 to 3, the concentrations of butyric acid were 205.86 μg/g in Group A, 288.09 μg/g in Group B (increase to approximately 1.4 times of that in Group A), 314.59 μg/g in Group C (increase to approximately 1.5 times of that in Group A), 754.17 μg/g in Group D (increase to approximately 3.7 times of that in Group A), and 2005.26 μg/g in Group E (increase to approximately 10 times of that in Group A). Moreover, the amounts of butyric acid was 781.14 μg in Group A, 1069.37 μg in Group B (increase to approximately 1.4 times of that in Group A), 1107.92 μg in Group C (increase to approximately 1.4 times of that in Group A), 2891.65 μg in Group D (increase to approximately 3.6 times of that in Group A), and 11943.88 μg in E group (increase to approximately 14.5 times of that in Group A). Accordingly, the concentrations and the amounts of butyric acid were markedly increased in any of Groups B to E in comparison with those in Group A.

The concentrations and the amounts of acetic acid were also significantly increased in Groups B, C, D, and E in comparison with those in Group A. Meanwhile, the concentrations and the amounts of propionic acid and isovaleric acid were not significantly different in Groups B, C, D, and E in comparison with those in Group A. The concentrations and the amounts of isobutyric acid and valeric acid were significantly decreased in Groups B, C, D, and E in comparison with those in Group A.

From this result, it was revealed that the concentration and the amount of cecal butyric acid were increased in the rats given intake of the feed with 1-kestose. Accordingly, it was revealed that intestinal butyric acid increases upon the intake of 1-kestose by humans and animals. Moreover, it was revealed that preferable intake of 1-kestose in human adult is equal to or more than 0.04 g/kg body weight per day to increase intestinal butyric acid since prominent increase in concentration and amount of butyric acid was found in rats with daily intake of 0.27 g/kg body weight of kestose (Group B).

### <Example 2> Study on Amount of Intestinal Microorganism Upon Intake of 1-Kestose

The amounts of the following 4 microorganisms in the cecal contents of rats in Groups A and E described in Example 1 (1) were measured by real-time PCR.

1. The genus Bifidobacterium,

2. Clostridium cluster-XIVa and Clostridium cluster-XIVb (a group of butyric acid-producing bacteria),

3. The genus Lactobacillus

4. Akkermansia muciniphila

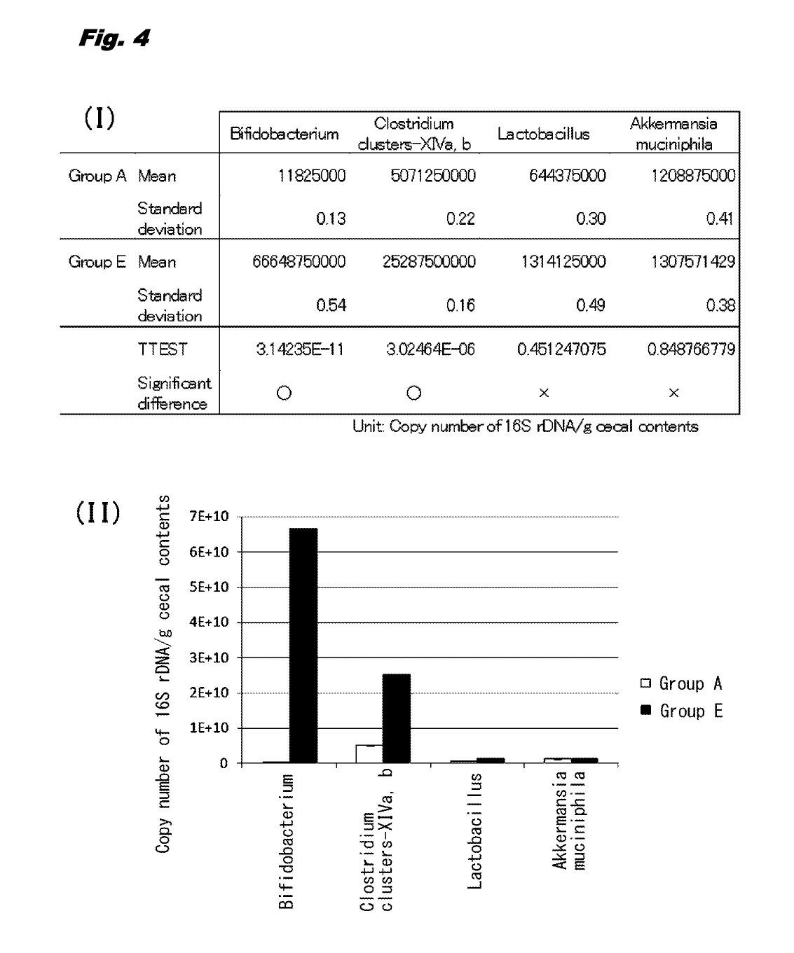

Specifically, the cecal contents of Groups A and E were disrupted with the bead beating system “FastPrep FP100A” (MP Biomedicals, LLC.). Subsequently, genomic DNA was extracted with the nucleic acid extraction instrument “Magtration® System 12GC” and its exclusive reagent “MagDEA® DNA 200” (Precision System Science Co., Ltd.) according to the instructions. Using this genomic DNA as a template, the real-time PCR was performed with primers specific for the 16S rDNAs of the aforementioned 4 microorganisms, the real-time PCR reagent “SYBR® Premix Ex Taq II (Tli RNaseH Plus)” (Takara), and the real-time PCR device “Rotor-Gene Q” (QIAGEN N.V.) and the copy numbers of the 16S rDNA from each microorganism per 1 g of the cecal contents were determined. The means and standard deviation of the copy numbers were calculated for each group and illustrated in bar graphs. The results are shown in FIG. 4.

The sequences of the primers specific for 16S rDNA of each microorganism are listed below.

1. Bifidobacterium
Forward primer,
(SEQ ID NO: 1)
GATTCTGGCTCAGGATGAACGC
Reverse primer,
(SEQ ID NO: 2)
CTGATAGGACGCGACCCCAT
2. Clostridium cluster XIVa and XIVb (a group of
butyric acid-producing bacteria)
Forward primer,
(SEQ ID NO: 3)
GAWGAAGTATYTCGGTATGT
Reverse primer,
(SEQ ID NO: 4)
CTACGCWCCCTTTACAC
3. Lactobacillus
Forward primer,
(SEQ ID NO: 5)
CACAATGGACGMAAGTCTGATG
Reverse primer,
(SEQ ID NO: 6)
CGCCACTGGTGTTCTTCCAT
4. Akkermansia muciniphila
Forward primer,
(SEQ ID NO: 7)
CAGCACGTGAAGGTGGGGAC
Reverse primer,
(SEQ ID NO: 8)
CCTTGCGGTTGGCTTCAGAT

The standard curve was made from the result of real-time PCR for each microorganism under the same conditions using a plasmid DNA in which a part of the following sequence is incorporated as a template.

1. The genus Bifidobacterium; 16S rDNA sequence (SEQ ID NO: 9) of B. longum subsp. longum JCM 1217T

2. Clostridium clusters-XIVa and -XIVb (a group of butyric acid-producing bacteria); 16S rDNA sequence (SEQ ID NO: 10) of Clostridium clostridioforme JCM1291T

3. The genus Lactobacillus; 16S rDNA sequence (SEQ ID NO: 11) of L. casei JCM 1134T

4. Akkermansia muciniphila; 16S rDNA sequence (SEQ ID NO: 12) of A. muciniphila ATCC BAA-835T

As illustrated in FIG. 4, the copy number of 16S rDNA from Clostridium cluster XIV (a group of butyric acid-producing bacteria) per 1 g of the cecal contents was 5071250000 (about 0.5E+10) in Group A and 25287500000 (about 2.5E+10) in Group E. Accordingly, the copy number of 16S rDNA from the group of butyric acid-producing bacteria in Group E was approximately 5 times of that in Group A and significantly increased.

The copy number of 16S rDNA from the genus Bifidobacterium conventionally known to be increased in the intestines by intake of kestose (for example, Japanese Patent No. 4669235) was also significantly increased in Group E in comparison with that in Group A. Meanwhile, the copy numbers of 16S rDNA from Lactobacillus and Akkermansia muciniphila did not have significant difference between Group E and Group A.

From this result, it was revealed that the number of cecal butyric acid-producing bacteria was increased in the rats given intake of the feed with 1-kestose. Accordingly, it was revealed that intestinal butyric acid-producing bacteria proliferate upon intake of 1-kestose by humans and animals.

1-8.

9. A method for proliferating butyric acid-producing bacteria, comprising the step of adding 1-kestose to a medium containing butyric acid-producing bacteria to culture the butyric acid-producing bacteria.

10. A method for treatment or prevention of intestinal inflammation, fatty liver, diabetes, colorectal cancer, or obesity, comprising the step of increasing the amount or the concentration of butyric acid or the number of butyric acid-producing bacteria in intestines in a human or an animal having or being at risk of having intestinal inflammation, fatty liver, diabetes, colorectal cancer, or obesity by letting take the human or the animal 1-kestose.

11. A method for producing a pharmaceutical preparation or a health food for treatment or prevention of intestinal inflammation, fatty liver, diabetes, colorectal cancer, or obesity using 1-kestose.


SEQUENCE LISTING (12 sequences)

SEQ ID NO: 1 - DNA, 22 bp, Artificial Sequence
  forward primer
gattctggctcaggatgaacgc

SEQ ID NO: 2 - DNA, 20 bp, Artificial Sequence
  reverse primer
ctgataggacgcgaccccat

SEQ ID NO: 3 - DNA, 20 bp, Artificial Sequence
  forward primer
gawgaagtatytcggtatgt

SEQ ID NO: 4 - DNA, 17 bp, Artificial Sequence
  reverse primer
ctacgcwccctttacac

SEQ ID NO: 5 - DNA, 22 bp, Artificial Sequence
  forward primer
cacaatggacgmaagtctgatg

SEQ ID NO: 6 - DNA, 20 bp, Artificial Sequence
  reverse primer
cgccactggtgttcttccat

SEQ ID NO: 7 - DNA, 20 bp, Artificial Sequence
  forward primer
cagcacgtgaaggtggggac

SEQ ID NO: 8 - DNA, 20 bp, Artificial Sequence
  reverse primer
ccttgcggttggcttcagat

SEQ ID NO: 9 - DNA, 1507 bp, Bifidobacterium longum subsp. longum JCM 1217T
gggtttcgattctggctcaggatgaacgctggcggcgtgcttaacacatgcaagtcgaacgggatccatcaagcttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctgga

SEQ ID NO: 10 - DNA, 1493 bp, Clostridium clostridioforme JCM 1291T
  misc_feature (1123)..(1124): n is a, c, g, or t
agagtttgatcctggctcaggatgaacgctggcggcgtgcctaacacatgcaagtcgaacgaagcaattaagatgaagttttcggatggaatcttgattgactgagtggcggacgggtgagtaacgcgtggataacctgcctcacactgggggataacagttagaaatgactgctaataccgcataagcgcacagtgccgcatggcagtgtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgccgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatccccctgacgggccggtaacgcggcctttccttcgggacaggggagacaggtggtgcatggttgtcgtcagctcgtgtcgtgagatgttgggttaagtcccgcaacgagcgcaacccttatccttagtagccagcasgtaragnngggcactctagggagactgccagggataacctggaggaaggtggggatgacgtcaaatcatcatgccccttatgatttgggctacacacgtgctacaatggcgtaaacaaagggaagcgagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaaggcggggcaggtaactggggtgaagtcgtaacaaggtaacc

SEQ ID NO: 11 - DNA, 1522 bp, Lactobacillus casei JCM 1134T
gatgaacgctggcggcgtgcctaatacatgcaagtcgaacgagttttggtcgatgaacggtgcttgcactgwgattcracttaaaacgagtggcggacgggtgagtaacacgtgggtaacctgcccttaagtgggggataacatttggaaacagatgctaataccgcataaatccaagaaccgcatggttcttggctgaaagatggcgtcaagctatcgcttttggatggacccgcggcgtattagctagttggtgaggtaacggctcaccaaggcgatgatacgtagccgaactgagaggttgatcggccacattgggactgagacacggcccaaactctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcattgagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacc

SEQ ID NO: 12 - DNA, 1505 bp, Akkermansia muciniphila ATCC BAA-835T
agagtttgattctggctcagaacgaacgctggcggcgtggataagacatgcaagtcgaacgagagaattgctagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgaagtatctgaagccaaccgcaaggaggcagggtcctaaggtgagactggtaactgggatgaagtcgtaacaaggtagccgtaggggaacctgcggctggatcacctccttt